(19) United States
(12) Patent Application Publication
(10) Pub. No.: US 2026/0174317 A1
(43) Pub. Date: Jun. 25, 2026

(54) INFINITELY VARIABLE COMBINED ROTARY TISSUE RETRACTOR, CAMERA HOLDER, AND AIR OR FLUID DELIVERY DEVICE FOR A VIDEOSCOPE OR ENDOSCOPE

(71) Applicant: QUALITY ASPIRATORS, INC., Duncanville, TX (US)

(72) Inventor: Stephen K. Harrel, Dallas, TX (US)

(21) Appl. No.: 18/987,737

(22) Filed: Dec. 19, 2024

(51) Int. Cl. A61B 1/05; A61B 1/015; A61B 1/12

(52) U.S. Cl. CPC A61B 1/05; A61B 1/015; A61B 1/123

(57) ABSTRACT

A surgical device, comprising a handle configured to be held by a user, a moveable tip coupled to the handle and a camera device disposed at the tip, wherein a field of view of the camera device can be radially adjusted.

### TECHNICAL FIELD

The present disclosure relates generally to surgical equipment, and more specifically to an infinitely variable combined rotary tissue retractor, camera holder, and air or fluid delivery device for a videoscope or endoscope.

### BACKGROUND OF THE INVENTION

There are many surgical devices that are needed for surgical procedures. While these can be helpful, it can also result in too many devices and clutter, which can lead to complications.

### SUMMARY OF THE INVENTION

A surgical device is disclosed that includes a handle that is configured to be held by a user. A moveable tip is coupled to the handle, and a camera device is disposed at the tip, wherein a field of view of the camera device can be radially adjusted by rotating the movable tip around the camera.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE SEVERAL VIEWS OF THE DRAWINGS

Aspects of the disclosure can be better understood with reference to the following drawings. The components in the drawings may be to scale, but emphasis is placed upon clearly illustrating the principles of the present disclosure. Moreover, in the drawings, like reference numerals designate corresponding parts throughout the several views, and in which:

FIG. 1 is a side view of a camera holder handle, in accordance with an example embodiment of the present disclosure;

FIG. 2 is an isometric view of a camera holder tip, in accordance with an example embodiment of the present disclosure;

FIG. 3 is a side view of a camera holder tip, in accordance with an example embodiment of the present disclosure;

FIG. 4 is an isometric view of a camera holder with a tip, in accordance with an example embodiment of the present disclosure; and

FIG. 5 is a side view of a camera holder with a tip, in accordance with an example embodiment of the present disclosure.

### DETAILED DESCRIPTION OF THE INVENTION

In the description that follows, like parts are marked throughout the specification and drawings with the same reference numerals. The drawing figures may be to scale and certain components can be shown in generalized or schematic form and identified by commercial designations in the interest of clarity and conciseness.

With current videoscopes and endoscopes used for surgical and non-surgical periodontal/dental procedures, it is necessary to utilize multiple different camera or endoscope fiber optic lead holders to visualize different areas, such as on a tooth or in different areas of a surgical site. As used herein, these various locations will be referred to as surgical sites, even for pre-surgical or non-surgical uses, and the term “surgical” should not be limiting to the scope or use of the disclosed innovations. As an example, to visualize a single tooth during non-surgical periodontal therapy, it is necessary to move the camera to 4 different specialized holding devices. This equipment limitation requires the operator to have multiple devices (handpieces), requires significant time to stop the procedure and change handpieces, and puts the camera or fiber optic lead at risk of damage.

The present disclosure is directed to a single handpiece with a tip that is easily rotated around the camera or fiber optic lead, which allows the operator to visualize all sides of a tooth or area of treatment by adjusting (rotating) a knurled knob. This procedure can be performed easily while the operator is working and eliminates the need to remove the camera from the treatment site and change handpieces, thus saving treatment time. This procedure also allows treatment to proceed seamlessly (without interruption) and protects the camera or fiber optic lead. In addition, the present disclosure allows the device to deliver air or a liquid such as water or saline to the operating site, to keep the camera free of debris and to flush debris and blood from the treatment area. It can be advantageously combined with suction, or a suction device can be used independently. The disclosed device can be made or used in a manner like that described and disclosed in U.S. Pat. No. 8,096,944, which is hereby incorporated by reference for all purposes as if set forth herein in its entirety.

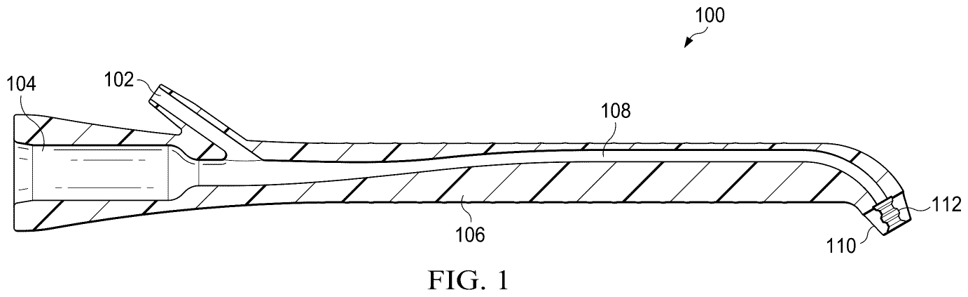

FIG. 1 is a side view of a camera holder handle 100, in accordance with an example embodiment of the present disclosure. Camera holder handle 100 includes inlet 102, camera port 104, body 106, channel 108, tip 110 and connector 112, each of which can be implemented in a polymer, a plastic, a metal material, a composite material or other suitable materials.

Inlet 102 is configured to allow an air source, a gas source, a water source, a saline source or other fluid sources to be coupled to camera holder handle 100, such as to allow a gas or fluid to be provided during a surgical procedure or operation. In one example embodiment, inlet 102 can be used to provide a gas for one aspect of a procedure and a liquid for another aspect of the procedure. Likewise, inlet 102 can be plugged where suitable, multiple inlets 102 can be provided or other suitable embodiments can also or alternatively be used.

Camera port 104 is configured to allow a miniature camera with a lens to be inserted into camera holder handle 100. In an example embodiment, the miniature camera can have a semiconductor imager with a predetermined number of pixels and can be self-focusing, can have an analog imager or analyzer, or can have other devices. The diameter of the miniature camera must be smaller than the minimum diameter of camera port 104 or channel 108, and can be a bronchoscope, a colonoscope, an endoscope, a duodenoscope, a gastroscope, a cystoscope, a fiberscope, a rhinolaryngoscope or other suitable devices. The miniature camera provides analog or digital signals representative of the image captured or other suitable signals, such as through an electrical cable, a wireless connection, a fiber optic connection or in other suitable manners, to remotely located image processing equipment or other data processing equipment. The camera can include a lens that is positioned beyond the end of the tip 110. The other end of the electrical cable carrying analog, digital, optical or other signals output from the camera can be connected to an imaging apparatus, a data processing apparatus or other suitable apparatuses for processing the analog, digital or optical signals and presenting the same on a suitable monitor or screen for display. The camera can also be equipped with one or more light emitting devices to illuminate the area to be imaged. Signals driving the illumination devices can be carried by conductors disposed in channel 108.

Body 106 can be formed from a polymer, a plastic, a metal material, a composite material or other suitable materials, using a suitable manufacturing process. In one example embodiment, body 106 can be formed by injection molding, computer-aided manufacturing or in other suitable manners. Body 106 can be rigid for applications that require a rigid body and can be flexible for applications that require a flexible body. The outer surface of body 106 can be treated to improve the ability to sterilize body 106 and to maintain its sterility, to clean body 106 and for other suitable purposes.

Channel 108 is disposed within body 106, and has a shape and contour to facilitate installation, removal and use of camera holder handle 100. In one example embodiment, the camera or other device used with camera holder handle 100 can have a larger outside diameter than the associated electrical or fiber optic lead, such that additional space can be provided for delivery of gas or liquids.

Tip 110 can include a structure that allows a camera holder tip to be inserted or otherwise attached to camera holder handle 100. In one example embodiment, tip 110 can be configured to protect connector 112, which can rotatably lock with a camera holder tip to allow the camera holder tip to rotate continuously within a range of angular locations. In this manner, the camera holder tip is infinitely variable to allow a practitioner to orient the camera as needed to provide an optimal angle.

In operation, camera holder handle 100 allows a camera to be used for a surgical operation without the need for multiple different camera holder devices. The prior art could require 4 or more camera holders to allow a practitioner to obtain clear views of teeth, cysts, sores, incisions, polyps or other structures, which would otherwise require the camera to be repeatedly removed and reinstalled in different holders. Such additional handling increases the costs, risks of damage to the equipment, time required and also risks losing sight of an important area that requires further investigation. In this manner, camera holder handle 100 provides novel features and functionality that allow a practitioner to obtain data that was not previously obtainable using prior art devices.

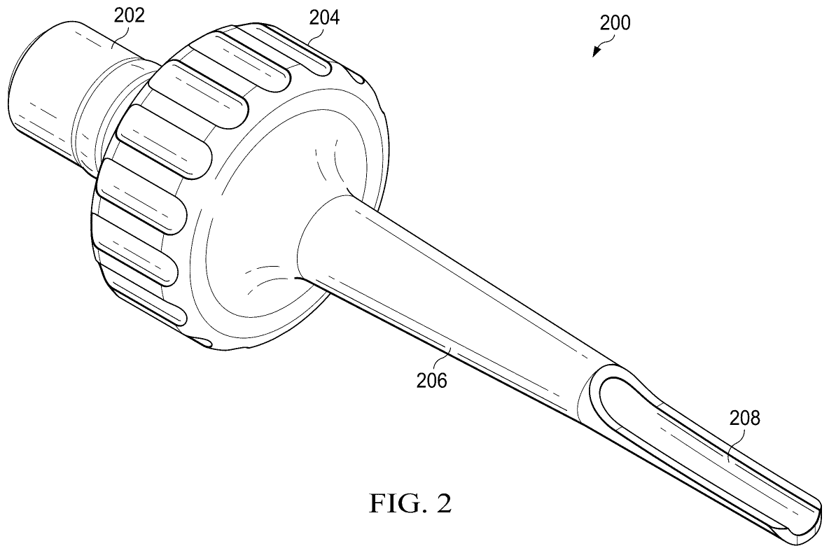

FIG. 2 is an isometric view of a camera holder tip 200, in accordance with an example embodiment of the present disclosure. Camera holder tip 200 includes connector 202, adjuster 204, stem 206 and support 208, each of which can be implemented in a polymer, a plastic, a metal material, a composite material or other suitable materials.

Connector 202 can be configured to connect camera holder tip 200 to camera holder handle 100 or other suitable devices. In one example embodiment, connector 202 can include one or more mating devices that form a mechanical connection with a fluid barrier, so as to allow a camera lead to be inserted through a channel in connector 202, to allow a fluid to be provided through camera holder tip 200 to a surgical site or to clean a camera lens, or for other suitable purposes.

Adjuster 204 can be configured to allow a user to adjust an angle of view for a camera that is disposed within camera holder tip 200, such as by rotating the adjuster circumferentially. While adjuster 204 is shown as a ridged cylinder, it can also or alternatively have other configurations, such as a locking device to hold adjuster 204 in a specific location, a fine tune control or other suitable configurations. In one example embodiment, the camera can have a 360-degree field of view, and the support 208 can be used to restrict a suitable portion of the field of view, such as between 30 degrees and 330 degrees, depending on the application. For example, some applications may benefit from a 160 to 200 degree field of view, whereas others may require a greater or smaller field of view.

Stem 206 can be configured to extend for a predetermined distance, as a function of the application. In one example embodiment, stem 206 can have a length that is suitable for dental procedures, ear procedures, nose procedures, throat procedures or other suitable procedures that require a rigid structure. In an alternative embodiment, stem 206 can have an extended flexible structure, such as for endoscopic procedures or other suitable procedures that require a flexible structure.

Support 208 can be configured to support a miniature camera or diagnostic device, which can be disposed on a lead, can be wireless or can otherwise be in data communication with a system or device that can receive and process data generated by the camera or other diagnostic device. While support 208 is shown as a partial segment extending from stem 206, it can also or alternatively be a support structure, a clear tube, one or more penetrations in a tube or other suitable structures. Support 208 can also or alternatively be configured as a support for the camera or fiber optic lead if the camera is to extend beyond the fully enclosed portion of the tip holder, such as for treatment, if the camera needs to be cleaned with saline gauze, or for other suitable purposes. Support 208 can also or alternatively be used as a retractor. The operator can place the round/shield side of the tip against the soft tissue, either surgically incised tissue or the edge of the gum during nonsurgical procedures, to push it away an create a space so the treatment site can be visualized.

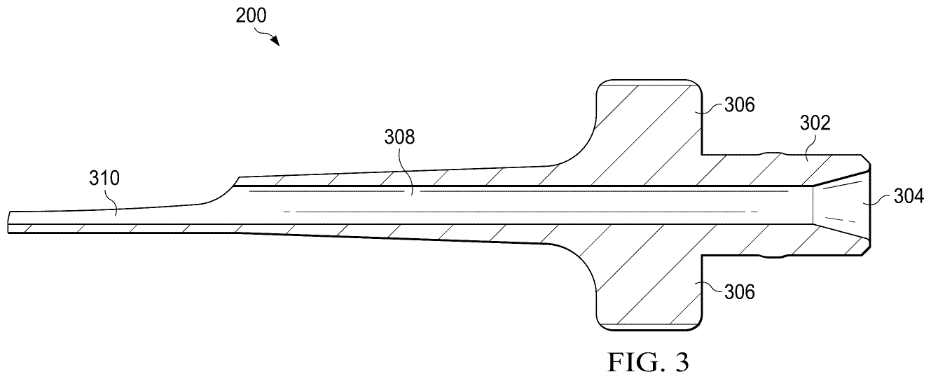

FIG. 3 is a side view of a camera holder tip 200, in accordance with an example embodiment of the present disclosure. Camera holder tip 200 includes connector 302, inlet 304, adjuster 306, channel 308 and support 310, each of which can be implemented in a polymer, a plastic, a metal material, a composite material or other suitable materials.

Connector 302 can be configured to connect camera holder tip 200 to camera holder handle 100 or other suitable devices. In one example embodiment, connector 302 can include one or more mating devices that form a mechanical connection with a fluid barrier, so as to allow a camera lead to be inserted through a channel in connector 302, to allow a fluid to be provided through camera holder tip 200 to a surgical site or to clean a camera lens, or for other suitable purposes.

Inlet 304 can be configured to interface with connector 112 or other suitable camera holder handles 100. In one example embodiment, inlet 304 can mate with connector 112 to form a mechanical connection with a fluid barrier, so as to allow a camera lead to be inserted through channel 308, to allow a fluid to be provided through support 310 to a surgical site or to clean a camera lens, or for other suitable purposes.

Adjuster 306 can be configured to allow a user to adjust an angle of view for a camera that is disposed within camera holder tip 200, such as by rotating adjuster 306 circumferentially. While adjuster 306 is shown as a ridged cylinder, it can also or alternatively have other configurations, such as a locking device to hold adjuster 306 in a specific location, a fine tune control or other suitable configurations.

Channel 308 is disposed within a body of camera holder tip 200, and has a shape and contour to facilitate installation, removal and use of a camera. In one example embodiment, the camera or other device used with camera tip 200 can have a larger outside diameter than the associated electrical or fiber optic lead while being smaller than the inside diameter of channel 308, such that additional space can be provided for delivery of gas or liquids while the lead is disposed in channel 308.

Support 310 can be configured to support a miniature camera or diagnostic device, which can be disposed on a lead, can be wireless or can otherwise be in data communication with a system or device that can receive and process data generated by the camera or other diagnostic device. While support 310 is shown as a partial segment extending from channel 308, it can also or alternatively be a support structure, a clear tube, one or more penetrations in a tube or other suitable structures.

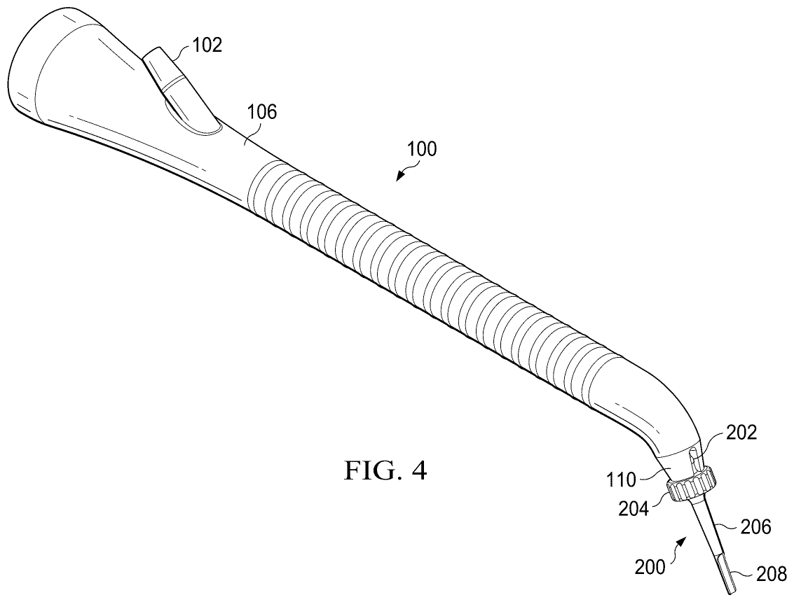

FIG. 4 is an isometric view of a camera holder 100 with a tip 200, in accordance with an example embodiment of the present disclosure. In addition to the components previously described, ridges 402 are disposed along the outer surface of body 106, and slot 404 is disposed in tip 110. Stem 206 can be rotated so as align support 208 in a suitable orientation, to allow image data or other suitable data to be generated.

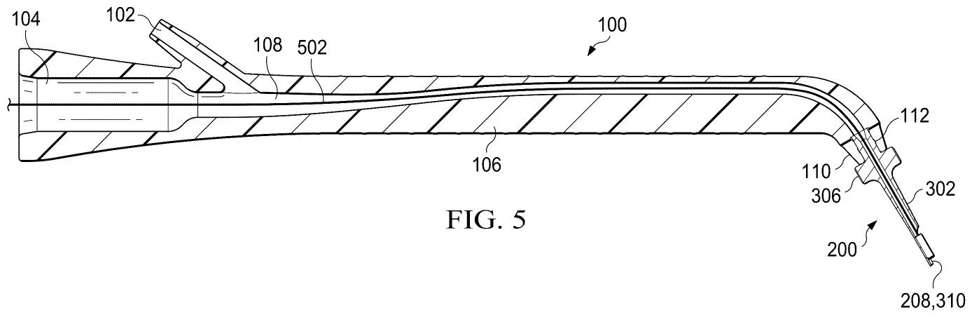

FIG. 5 is a side view of a camera holder 100 with a tip 200, in accordance with an example embodiment of the present disclosure. In addition to the components previously described, camera 502 is disposed within channel 108. Camera 502 can be a videoscope imaging system with a CMOS imager, such as a compact HD board-level camera, model OV6946 or OV6948, available from Myriad Fiber Imaging of Dudley, MA or other suitable systems or imaging devices. Adjuster 306 can be rotated so as align support 208/310 in a suitable orientation, to allow image data or other suitable data to be generated.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. A software system is typically created as an algorithmic source code by a human programmer, and the source code algorithm is then compiled into a machine language algorithm with the source code algorithm functions, and linked to the specific input/output devices, dynamic link libraries and other specific hardware and software components of a processor, which converts the processor from a general purpose processor into a specific purpose processor. This well-known process for implementing an algorithm using a processor should require no explanation for one of even rudimentary skill in the art. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. A system can receive one or more data inputs, such as data fields, user-entered data, control data in response to a user prompt or other suitable data, and can determine an action to take based on an algorithm, such as to proceed to a next algorithmic step if data is received, to repeat a prompt if data is not received, to perform a mathematical operation on two data fields, to sort or display data fields or to perform other suitable well-known algorithmic functions. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

What is claimed is:

1. A surgical device, comprising:
a handle configured to be held by a user;
a moveable tip coupled to the handle and configured to form a shield around a camera; and
a camera device disposed at the tip, wherein a field of view of the camera device can be radially adjusted by adjusting the moveable tip.

2. The surgical device of claim 1 wherein the camera device is configured to be extended from the moveable tip.

3. The surgical device of claim 1 wherein the moveable tip forms a circumferential shield.

4. The surgical device of claim 1 wherein the moveable tip forms a circumferential shield that restricts the field of view of the camera device.

5. The surgical device of claim 1 wherein the moveable tip forms a circumferential shield that restricts the field of view of the camera device to approximately 180 degrees.

6. The surgical device of claim 1 wherein the handle comprises a port for a fluid or gas.

7. The surgical device of claim 1 wherein the handle comprises a shaft holding a lead of the camera device, wherein the lead is disposed in a channel of the shaft.

8. The surgical device of claim 1 wherein the handle comprises a port for a fluid or gas, and a shaft holding a lead of the camera device, wherein the lead is disposed in a channel of the shaft and the channel is configured to conduct the fluid or the gas while containing the lead.

9. A method for using a surgical imaging device, comprising:
inserting a camera device into a channel of a handle until an imaging device of the camera device is exposed at a moveable tip; and
radially adjusting a field of view of the camera device by adjusting the moveable tip.

10. The method of claim 9 further comprising extending the camera device from the moveable tip.

11. The method of claim 9 wherein radially adjusting the field of view of the camera comprises radially adjusting a circumferential shield of the moveable tip.

12. The method of claim 9 further comprising using the moveable tip as a retractor.

13. The method of claim 9 further comprising radially adjusting the field of view of the camera to an area of approximately 180 degrees.

14. The method of claim 9 further comprising cleaning the camera device using a fluid or gas disposed through the channel of the handle.

15. The method of claim 9 further comprising coupling a source of fluid or gas to a port of the handle.

16. The method of claim 9 further comprising:
placing the moveable tip at a first treatment location;
generating first image data at the first location;
radially adjusting the field of view of the camera; and
generating second image data at the first location.
